(19) United States
(12) Patent Application Publication
(10) Pub. No.: US 2019/0381216 A1
(43) Pub. Date: Dec. 19, 2019

(54) TUBE FOR MEDICAL INSTRUMENTS

(71) Applicant: OLYMPUS CORPORATION, Tokyo (JP)

(72) Inventor: Takaaki HANAI, Tokyo (JP)

(73) Assignee: OLYMPUS CORPORATION, Tokyo (JP)

(21) Appl. No.: 16/552,240

(22) Filed: Aug. 27, 2019

Related U.S. Application Data
(63) Continuation of application No. PCT/JP2018/018893, filed on May 16, 2018.

(30) Foreign Application Priority Data
Jun. 1, 2017 (JP) 2017-109260

(51) Int. Cl. A61L 29/04; A61B 1/018; A61M 25/00

(52) U.S. Cl. CPC A61L 29/042; A61B 1/018; A61M 25/0053; A61M 2205/0216; A61L 29/041; A61M 2025/0046; A61M 25/0045

(57) ABSTRACT

A tube for medical instruments according to the present invention includes: a tubular member that is made of resin, a linear member that includes a metal wire member and a resin coating film that covers the metal wire member and that surrounds the tubular member outside the tubular member, and a polymeric elastomer layer that is laminated on an outer circumferential surface of the tubular member such that the polymeric elastomer layer fills in at least a part of an outer peripheral portion of the linear member in a circumferential direction over a longitudinal direction of the linear member.

### CROSS REFERENCE TO RELATED APPLICATIONS

This application is a continuation application based on a PCT Patent Application No. PCT/JP2018/018893, filed on May 16, 2018, whose priority is claimed on Japanese Application No. 2017-109260, filed on Jun. 1, 2017, the entire content of which are hereby incorporated by reference.

### BACKGROUND OF THE INVENTION

### Field of the Invention

The present invention relates to a tube for medical instruments.

### Description of the Related Art

Tubes for medical instruments are used by being inserted into curved tubes such as channels for endoscopes, for example. Treatment tools or the like are inserted into and removed from an inside of the tubes for medical instruments in a state in which the tubes are inserted into curved channels for endoscopes, for example.

The tubes for medical instruments used in this manner are configured by being reinforced with a reinforcing member with flexibility in order to be able to maintain tubular shapes into which treatment tools can be smoothly inserted even when the tubes are bent, in many cases.

For example, a treatment tool insertion channel disclosed in Japanese Unexamined Patent Application, First Publication No. H3-205022 includes a tube main body with an inner surface is made of urethane resin coated with fluorine resin, a net that is arranged outside the tube main body and is configured of stainless wires, and a coating layer that is made of urethane resin and covers the net to prevent the net from being exposed to the outside.

For example, a flexible tube for an endoscope disclosed in Japanese Unexamined Patent Application, First Publication No. S58-195538 includes a flex that is formed by winding a band-like metal thin plate in a spiral tube shape, a tubular blade that is fitted to the outer periphery of the flex, an adhesive layer that is applied to the outer circumferential surface of the blade and penetrates up to an inner surface of the blade, and an external skin that is formed into a tubular shape and is made of thermoplastic resin or synthetic resin with an inner surface to which the adhesive layer is adhered and secured.

For example, a tube for an endoscope disclosed in Japanese Unexamined Patent Application, First Publication No. 2010-29435 includes a tube main body that is made of fluorine resin, a reinforcing tape, and an external skin that is made of polyurethane and covers the tube main body on the reinforcing tape. The reinforcing tape includes a reinforcing layer that includes reinforcing fibers disposed in a circumferential direction and an axial direction and made of hard resin, and an adhesive layer. The reinforcing tape adheres to the tube main body with the adhesive layer in a state in which the reinforcing tape is wrapped around the outer circumferential surface of the tube main body.

### SUMMARY

A tube for medical instruments includes a tubular member that is made of resin, a linear member that includes a metal wire member and a resin coating film that covers the metal wire member and that is disposed so as to surround the tubular member outside the tubular member, and a polymeric elastomer layer that is laminated on an outer circumferential surface of the tubular member such that the polymeric elastomer layer fills in at least a part of a surface of the linear member in a circumferential direction over longitudinal direction of the linear member.

The linear member may form a net-shaped body.

The resin coating film may be made of fluorine resin.

The fluorine resin may contain polytetrafluoroethylene.

The fluorine resin may contain tetrafluoroethylene-perfluoroalkyl vinyl ether copolymer.

An inner circumferential surface of the tubular member may be configured of fluorine resin.

The polymeric elastomer layer may contain fluorine rubber.

### BRIEF DESCRIPTION OF THE DRAWINGS

FIG. 1 is a schematic partial sectional view showing a configuration example of a tube for medical instruments according to a first embodiment of the present invention.

FIG. 2 is a schematic sectional view showing a configuration example of a linear member that is used for the tube for medical instruments according to the first embodiment of the present invention.

FIG. 3 is a schematic view showing effects of the tube for medical instruments according to the first embodiment of the present invention.

FIG. 4 is a schematic view showing effects of a tube for medical instruments according to a comparative example.

FIG. 5 is a schematic partial sectional view showing a configuration example of a tube for medical instruments according to a second embodiment of the present invention.

FIG. 6 is a schematic view showing a test method for insertion durability evaluation conducted on evaluation samples (tubes for medical instruments) in an example and a comparative example.

FIG. 7 is a schematic view showing a test method for kink resistance evaluation conducted on evaluation samples (tubes for medical instruments) in an example and a comparative example.

FIG. 8 is a schematic view showing a test method for flexibility evaluation conducted on evaluation samples (tubes for medical instruments) in an example and a comparative example.

### DETAILED DESCRIPTION OF THE PREFERRED EMBODIMENTS

Hereinafter, embodiments of the invention will be described with reference to accompanying drawings. In all the drawings, the same reference numerals will be given to the same or corresponding members in all the drawings in different embodiments, and duplicated description will be omitted.

### First Embodiment

A tube for medical instruments according to a first embodiment of the present invention will be described.

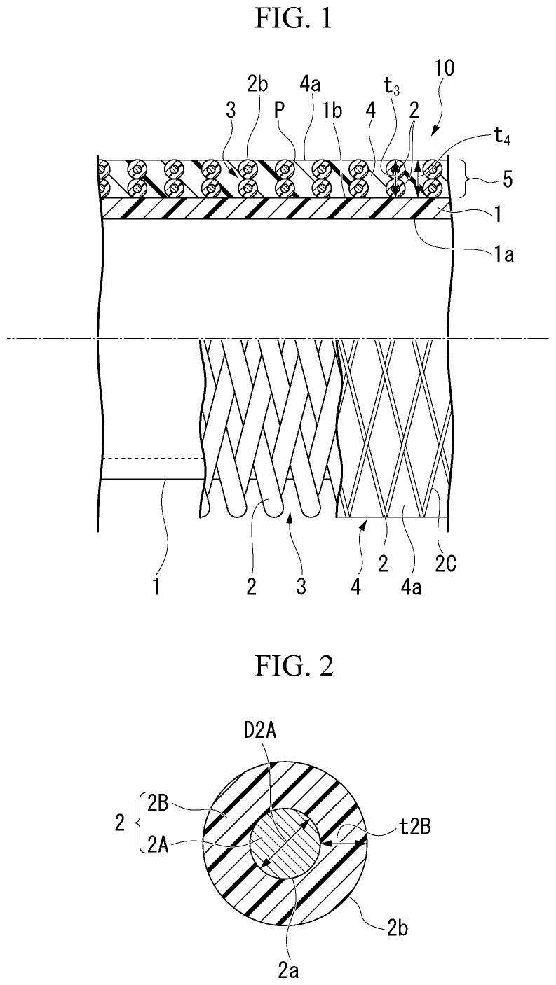

FIG. 1 is a schematic partial sectional view showing a configuration example of a tube for medical instruments 10 according to a first embodiment of the present invention.

As shown in FIG. 1, the tube for medical instruments 10 according to the first embodiment includes an inner layer tube (tubular member) 1 and an outer layer portion 5.

The tube for medical instruments 10 is a flexible tube for medical instruments. A medical instrument with which the tube for medical instruments 10 is used is not limited. For example, the tube for medical instruments 10 may be used as a treatment tool channel into which a treatment tool or the like is inserted in an endoscope device.

For example, the tube for medical instruments 10 may be used as an air or water feeding tube, a catheter for a treatment tool, or the like.

The inner layer tube 1 is a tubular member that includes a through-hole formed therein such that it extends in a longitudinal direction and that is made of resin. An axial or tubular insertion member such as a treatment tool or a catheter, for example, can be inserted into an inner circumferential surface 1a that forms the through-hole.

As a material for the inner layer tube 1, an appropriate resin with which a flexibility required by the inner layer tube 1 can be obtained is used. In order to prevent abrasion of the inner circumferential surface 1a, a resin with satisfactory slipping properties is more preferably used as a material for the inner layer tube 1.

As a material for the inner layer tube 1, a resin with which satisfactory chemical resistance, biocompatibility, cleaning and sterilizing properties, air tightness, water tightness, and the like can be obtained in accordance with requirements for a medical instrument used is more preferably used.

Examples of the material for the inner layer tube 1 that may be used include polyethylene, polypropylene, polystyrene, polyvinyl chloride, polymethyl methacrylate, polymethyl acrylate, acrylonitrile-butadiene-styrene, acrylonitrile-styrene, polyvinyl alcohol, polyester, polyethylene terephthalate, polyurethane, polymethylpentene, brominated polyethylene, an ethylene-vinyl acetate copolymer, an ethylene-vinyl alcohol copolymer, an ethylene-methyl acrylate copolymer, and general-purposed plastics such as ionomers.

Examples of the material for the inner layer tube 1 that may be used include engineering plastics such as polycarbonate, polyacetal, polyamide, polybutylene terephthalate, polybutylene naphthalate, or polyethylene naphthalate.

Examples of the material for the inner layer tube 1 that may be used include super-engineering plastics such as polyphenylene sulfide, polyether imide, polysulfones, polyarylate, polyimide, polyethersulfones, polyamideimide, polyether ether ketone, polyallyl ether ketone, or polyethernitrile.

Examples of the material for the inner layer tube 1 that may be used include fluorine resins such as polytetrafluoroethylene (PTFE), ethylene-tetrafluoroethylene copolymer (ETFE), tetrafluoroethylene-hexafluoropropylene copolymer, polychlorotrifluoroethylene, tetrafluoroethylene-perfluoroalkyl vinyl ether copolymer (PFA), polyvinylidene fluoride, or a chlorotrifluoroethylene-ethylene copolymer.

Examples of the material for the inner layer tube 1 that may be used include a thermoplastic elastomer such as a urethane-based thermoplastic elastomer, an ester-based thermoplastic elastomer, an amide-based thermoplastic elastomer, a styrene-based thermoplastic elastomer, an olefin-based thermoplastic elastomer, a fluorine-based thermoplastic elastomer, or a vinyl chloride-based thermoplastic elastomer.

Each of the aforementioned materials may be used alone for the inner layer tube 1, or a plurality of materials among the aforementioned materials may be used in combination.

In a case in which a composite material is used for the inner layer tube 1, a material in which a plurality of materials are blended in a dispersed manner may be used as the composite material.

In the case in which a composite material is used for the inner layer tube 1, the plurality of materials may have a layered structure. For example, the inner layer tube 1 may be configured of a base material of a first material and a coating layer of a second material that is different from the first material. For example, at least either the inner circumferential surface 1a or an outer circumferential surface 1b of the inner layer tube 1 may be configured as the coating layer.

Among the aforementioned materials, a fluorine resin is more preferably contained in the material for the inner layer tube 1 in terms of having excellent slipping properties. PTFE and PFA, for example, in the fluorine resin are particularly preferably used since they have particularly excellent slipping properties.

A fluorine resin is also more preferably included in the material for the inner layer tube 1 in terms of having excellent chemical resistance against chemicals used for sterilization treatment or the like. PTFE and PEA, for example, in the fluorine resin are particularly preferably used since they have particularly excellent chemical resistance.

In a case in which the inner layer tube 1 includes a base material and a coating layer, for example, a material with excellent slipping properties and chemical resistance may be used for the coating layer.

The outer layer portion 5 includes a linear member 2 and a polymeric elastomer layer 4.

As shown in FIG. 2, the linear member 2 is configured of a metal wire (metal wire member) 2A and a resin coating film 2B.

As the metal wire 2A, a metal single wire such as a round wire (that is, a wire with a circular sectional shape) or a flat wire (that is, a strip-shaped wire with a rectangular sectional shape), for example, may be used.

For the metal wire 2A, a stranded wire obtained by single metal wires forming a strand may be used. The number of wires in the strand, the strand configuration, and the strand direction are not particularly limited.

In a case in which the metal wire 2A is formed as a wire strand, both the materials and the shapes of a plurality of wires that configure the wire strand may be the same, or at least one of them may be different. In the case in which the metal wire 2A is made of wire strand, an outer circumferential surface 2a represents an outer circumferential surface of the entire wire strand. That is, in the case in which the metal wire 2A is made of wire strand, the outer circumferential surface 2a is configured of the entire of each surfaces of a state in exposing to the outermost peripheries in each wire strands.

As a diameter D2A and a material of the metal wire 2A, an appropriate diameter and a material with which flexibility and strength required by a net-shaped body 3, which will be described later are satisfied are used.

For example, the diameter D2A of the metal wire 2A may be equal to or greater than 0.02 mm and equal to or less than 0.3 mm.

Examples of the material used for the metal wire 2A include copper, copper alloys, carbon steel (piano wire), stainless steel, titanium, titanium alloys, nickel titanium alloys, tungsten, tungsten alloys, nickel alloys, cobalt alloys, amorphous metal, and the like.

Examples of copper alloys include brass. Examples of titanium alloys include 64 titanium. Examples of tungsten alloys include a tungsten (W)-rhenium (Re) alloy. Examples of nickel alloys include a nickel (Ni)-chromium (Cr)-iron (Fe) alloy and a nickel-chromium-iron-niobium (Nb)-molybdenum (Mo) alloy. Examples of cobalt alloys include a cobalt (Co)-chromium alloy.

The material from which the metal wire 2A is made is more preferably a metal with excellent toughness that has been subjected to autoclave sterilization and does not easily corrode. Examples of the metal with excellent toughness that has experienced autoclave sterilization and is not easily corroded include stainless steel.

The resin coating film 2B covers the outer circumferential surface 2a of the metal wire 2A. The resin coating film 29 is coaxial with the metal wire 2A. An outer circumferential surface 2b of the resin coating film 2B configures the surface of the outermost periphery of the linear member 2.

For example, a film thickness t2B of the resin coating film 2B may be equal to or greater than 0.02 mm and equal to or less than 0.3 mm.

As the material for the resin coating film 2B, an appropriate resin with a smaller elastic modulus than that of the metal wire 2A is used. The material for the resin coating film 2B is more preferably a resin with satisfactory slipping properties with respect to the polymeric elastomer layer 4 which will be described later.

Since a part of the resin coating film 2B according to the first embodiment is exposed to the outer portion of the polymeric elastomer layer 4 as will be described later, it is more preferable to use a resin with satisfactory chemical resistance against chemicals used for sterilization treatment or the like as the material for the resin coating film 2B.

Specific examples of the material used for the resin coating film 2B include the resin materials and combinations of resin materials exemplified as examples for the material for the inner layer tube 1.

The surface of the resin coating film 2B more preferably contains a fluorine resin in terms of particularly satisfactory slipping properties with respect to the polymeric elastomer layer 4 which will be described later. PTFE or PFA, for example, in the fluorine resin are particularly preferably contained in the surface of the resin coating film 2B since they have particularly excellent slipping properties.

The resin coating film 2B is more preferably made of fluorine resin since fluorine resin has excellent chemical resistance against chemicals used for sterilization treatment or the like. PTFE and PFA are particularly preferably used as the material for the resin coating film 2B since they have particularly excellent chemical resistance.

The linear member 2 with such a configuration forms the net-shaped body 3 with a tubular shape that surrounds the inner layer tube 1 from the side of the outer periphery as schematically shown in FIG. 1.

How the manner in which the net-shaped body 3 is knitted or woven from the linear member 2 is not particularly limited. Examples of how the net-shaped body 3 is knitted or woven include plain weaving, twilling, satin weaving, a knotless net, and the like.

The net-shaped body 3 is configured such that gaps are generated between linear members 2 that are adjacent in a direction along the surface of the inner layer tube 1 such that the net-shaped body 3 is easily bent along with the inner layer tube 1.

An inner peripheral portion of the net-shaped body 3 may be separated from the outer circumferential surface 1b of the inner layer tube 1. However, FIG. 1 shows an example in which the inner peripheral portion of the net-shaped body 3 is in contact with the outer circumferential surface 1b of the inner layer tube 1.

The polymeric elastomer layer 4 is laminated on the outer circumferential surface 1b of the inner layer tube 1. The polymeric elastomer layer 4 covers the entire outer circumferential surface 1b of the inner layer tube 1 at least at a part of the tube for medical instruments 10, which is inserted into the medical instrument.

The polymeric elastomer layer 4 is formed to have such a layer thickness that at least a part of the outer circumferential surface 2b in a circumferential direction that forms the surface of the linear member 2 is buried along the entirety of the linear member 2 in the longitudinal direction. Here, “at least a part of the outer circumferential surface 2b in a circumferential direction that forms the surface of the linear member 2 is buried along the entirety of the linear member 2 in the longitudinal direction” means that the outer circumferential surface 2b is not exposed over the entire periphery and only a part thereof in the circumferential direction is exposed even if the outer circumferential surface 2b is exposed from the polymeric elastomer layer 4 entirely in the longitudinal direction, at a portion at which the linear member 2 is present.

As shown in FIG. 1, for example, only a part of the outer circumferential surface 2b of the linear member 2 in the circumferential direction is exposed at an exposed portion 2C of the linear member 2 of the polymeric elastomer layer 4.

With such a configuration, the polymeric elastomer layer 4 is filled between the linear members 2 that are adjacent in the direction along the outer circumferential surface 1b in the net-shaped body 3 in the tube for medical instruments 10 according to the first embodiment.

In a case in which the inner peripheral portion of the net-shaped body 3 is in contact with the outer circumferential surface 1b of the inner layer tube 1 as in the example shown in FIG. 1, for example, a layer thickness t₄ of the polymeric elastomer layer 4 is smaller than a thickness t₃ of the net-shaped body 3 on the assumption that the thickness of the net-shaped body 3 is represented as the thickness t₃. A parameter (t₃-t₄) that represents the exposure height of the net-shaped body 3 in a radial direction is less than the outer diameter of the linear member 2 that consists the net-shaped body 3.

It is more preferable that a half or more of the linear member 2 in the circumferential direction be in a state in which it is buried in the polymeric elastomer layer 4. In order to form such a buried state, it is only necessary for the parameter (t₃-t₄) to be less than ½ of the outer diameter of the linear member 2.

A material for the polymeric elastomer layer 4 is not particularly limited as long as the material is a polymeric elastomer with which satisfactory flexibility of the tube for medical instruments 10 is achieved and with which more satisfactory slipping properties with respect to the resin coating film 2B of the linear member 2 than that of the metal wire 2A is achieved. In a case in which the tube for medical instruments 10 is subject to sterilization treatment or is brought into contact with a chemical solution, a material with satisfactory chemistry resistance is more preferably used for the polymeric elastomer layer 4.

Examples of the material for the polymeric elastomer layer 4 that may be used include thermoplastic elastomers such as a urethane-based thermoplastic elastomer, an ester-based thermoplastic elastomer, an amide-based thermoplastic elastomer, a styrene-based thermoplastic elastomer, an olefin-based thermoplastic elastomer, a fluorine-based thermoplastic elastomer, and a vinyl chloride-based thermoplastic elastomer.

Examples of the material for the polymeric elastomer layer 4 that may be used include vulcanized rubber such as natural rubber, isoprene rubber, butadiene rubber, styrene-butadiene rubber, butyl rubber, ethylene-propylene rubber, chloroprene rubber, chlorosulfonated polyethylene rubber, nitrile rubber, silicone rubber, urethane rubber, acrylic rubber, and fluorine rubber.

Each of the aforementioned materials suitable for the polymeric elastomer layer 4 may be used alone, or some of them may be used in combination for the polymeric elastomer layer 4.

In a case in which a plurality of materials are combined, for example, the plurality of materials may be blended or may be used as a composite material with a layered structure. In the case in which a plurality of materials are combined, the plurality of materials may be used at different portions in the polymeric elastomer layer 4 in the longitudinal direction.

Further, each of the aforementioned materials may be used as a solid body or may be used as a foaming body. In a case in which each of the aforementioned materials is used in a foaming state, each material may be a foaming body of an independent air bubble or may be a foaming body of sequential air bubbles. In the case in which the polymeric elastomer layer 4 is configured of a foaming body, the flexibility of the tube for medical instruments 10 is further improved.

The polymeric elastomer layer 4 more preferably contains at least either fluorine-based thermoplastic elastomer or fluorine rubber, for example, among the aforementioned respective materials in terms of more satisfactory slipping properties with respect to the resin coating film 2B.

To manufacture the tube for medical instruments 10 with the aforementioned configuration, each of the inner layer tube 1 and the net-shaped body 3 is manufactured in advance.

The inner layer tube 1 may be manufactured through extrusion molding, for example. Surface treatment for satisfactory adhesiveness with the polymeric elastomer layer 4 may be performed on the outer circumferential surface 1b of the inner layer tube 1.

The net-shaped body 3 is manufactured by the linear members 2 being knitted or woven after the linear members 2 are manufactured. As an example of a method for manufacturing each linear members 2, a manufacturing method in which the metal wire 2A is dipped into a resin solution for forming the resin coating film 2B and the resin solution adhering to the metal wire 2A is then hardened may be employed.

If the inner layer tube 1 and the net-shaped body 3 are prepared, the net-shaped body 3 is disposed around the outer circumferential surface 1b of the inner layer tube 1.

Thereafter, a fluid body containing a resin constituent that serves as the polymeric elastomer layer 4 is applied so as to cover the net-shaped body 3 and the inner layer tube 1. The amount of applied fluid body is set to such an amount of application that the exposed portion 2C of the net-shaped body 3 is formed when the fluid body is hardened. Thereafter, the fluid body is solidified. However, in a case in which the polymeric elastomer layer 4 is configured of the foaming body, the fluid body is solidified and is subject to foaming treatment.

The tube for medical instruments 10 is manufactured in this manner.

Next, effects of the tube for medical instruments 10 will be described.

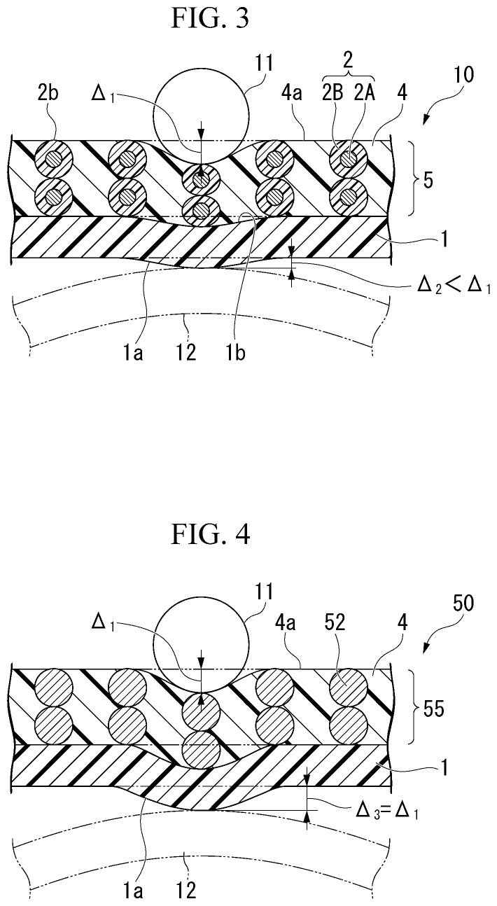

FIG. 3 is a schematic view showing effects of the tube for medical instruments 10 according to the first embodiment of the present invention. FIG. 4 is a schematic view showing effects of a tube for medical instruments 50 according to a comparative example.

In the tube for medical instruments 10, the net-shaped body 3 and the polymeric elastomer layer 4 are disposed in an outer periphery of the inner layer tube 1. The net-shaped body 3 is buried in the polymeric elastomer layer 4 except for the exposed portion 2C. Since the outer circumferential surface 1b of the inner layer tube 1 and the polymeric elastomer layer 4 are in close contact with each other, the net-shaped body 3 is disposed in the outer periphery of the inner layer tube 1 in a state in which the net-shaped body 3 is constrained by the polymeric elastomer layer 4.

However, the net-shaped body 3 is configured of the linear members 2, in each of which the outer circumferential surface 2a of the metal wire 2A is covered with the resin coating film 2B. Therefore, the linear members 2 that are in contact with each other in the net-shaped body 3 are in contact via the resin coating film 2B that is softer than the metal wire 2A. Therefore, if an external force is imparted on the net-shaped body 3, the thickness of the net-shaped body 3 (that is, the diameter of the net-shaped body 3 in the radial direction of the tube for medical instruments 10) is reduced at a portion at which the linear members 2 are superimposed on each other due to deformation of the resin coating film 2B.

In a case in which external force from an external pressing member 11 toward the inside is imparted on the tube for medical instruments 10 as shown in FIG. 3, for example, the linear members 2 are compressed in the radial direction (that is, the vertical direction in FIG. 3) due to deformation of the resin coating film 2B, and a projecting amount Δ₂ of the inner circumferential surface 1a of the inner layer tube 1 thus becomes smaller than a recessed amount Δ₁ even in a case in which the recessed amount in an outer circumferential surface 4a of the polymeric elastomer layer 4 is Δ₁. Therefore, rubbing abrasion of the inner circumferential surface 1a due to a treatment tool 12 is reduced when the treatment tool 12 is inserted into the inner layer tube 1 as compared with a case in which the projecting amount Δ₂ reaches the recessed amount Δ₁.

For example, the tube for medical instruments 50 according to the comparative example shown in FIG. 4 includes a metal wire 52 made of the same material as that of the metal wire 2A, instead of the linear members 2 in the tube for medical instruments 10. The outer diameter of the metal wire 52 is equal to the outer diameter of the linear members 2. If an external force from the external pressing member 11 toward the inside is imparted on the tube for medical instruments 50, the metal wire 52 is substantially not deformed in the radial direction, and the projecting amount Δ₃ of the inner circumferential surface 1a of the inner layer tube 1 thus reaches the recessed amount Δ₁ in the outer circumferential surface 4a of the polymeric elastomer layer 4. Therefore, since the projecting amount of the inner circumferential surface 1a becomes larger than that in the first embodiment in a case in which the treatment tool 12 is inserted into the inner layer tube 1, rubbing abrasion of the inner circumferential surface 1a due to the treatment tool 12 more significantly occurs.

If abrasion of the inner circumferential surface 1a advances, there is a concern that a hole may be opened in the inner layer tube 1. Even in a case in which no hole is opened, there is still a concern that kink may occur from an abrasion portion (that is, a portion at which abrasion has advanced in the inner circumferential surface 1a) in a case in which an external force is imparted on the tube for medical instruments since the strength of the abrasion portion is degraded.

Since the abrasion itself less occurs in the tube for medical instruments 10 according to the first embodiment than in the tube for medical instruments in the comparative example, the kink due to the abrasion portion is prevented from occurring.

Further, in a case in which the tube for medical instruments 10 is bent, a compression stress (that is, a stress generated on the inner side in the radial direction when bending in the axial direction is caused) or a tensile stress (that is, a stress generated on the outer side in the radial direction when bending in the axial direction is caused) is imparted on the linear members 2 at the outer layer portion 5 along the axial direction of the tube for medical instruments 10. The resin coating film 2B of each linear member 2 can be deformed in accordance with the respective stresses even in response to such stresses in the axial direction. Since an internal stress of the outer layer portion 5 is alleviated, the tube for medical instruments 10 can be easily bent and deformed. As a result, the flexibility of the tube for medical instruments 10 is improved.

Meanwhile, in the case of the tube for medical instruments 50 according to the comparative example, rigidity of an outer layer portion 55 is higher than rigidity of the outer layer portion 5 of the tube for medical instruments 10 since the metal wire 52 is substantially not deformed in response to the stresses in the axial direction. As a result, flexibility of the tube for medical instruments 50 is inferior to the flexibility of the tube for medical instruments 10.

Further, in a case in which the resin coating film 2B is formed of a material that is slippery with respect to the polymeric elastomer layer 4, slipping occurs between the outer circumferential surface 2b of each linear member 2 and the polymeric elastomer layer 4 in response to a stress generated at the outer layer portion 5. Since this leads to a decrease in resistance against bending of the tube for medical instruments 10, it becomes easier to bend the tube for medical instruments 10.

Further, if an internal stress of the outer layer portion 5 is released by slipping of the linear member 2 with respect to the polymeric elastomer layer 4, stress distribution in the outer layer portion 5 is equally leveled. This prevents kink that is caused by concentration of deformation at a part of the tube for medical instruments 10 due to localization of stress distribution in the outer layer portion 5 from occurring. As a result, the kink resistance of the tube for medical instruments 10 is improved.

In the tube for medical instruments 10, the exposed portion 2C of each linear member 2 is formed in the polymeric elastomer layer 4. An interface P between the linear member 2 and the polymeric elastomer layer 4 is linearly exposed at the exposed portion 2C. The interface P continues from the exposed portion 2C to the inner circumferential surface of the polymeric elastomer layer 4 along the outer circumferential surface 2b of the linear member 2. In a case in which the resin coating film 2B is configured of a material that easily slips with respect to the polymeric elastomer layer 4, and if an external force is imparted on the polymeric elastomer layer 4, the polymeric elastomer layer 4 easily peels off from the resin coating film 29 at the interface P.

With such a configuration, it is easy to inspect whether or not cracking or a through-hole (hereinafter, collectively referred to as a “hole”) crossing in the thickness direction of the inner layer tube 1, for example, is present.

Since a treatment tool or the like is inserted into and removed from the inner layer tube 1, and an external force caused by the treatment tool or the like is imparted thereon, or abrasion occurs therein, there is a case in which a hole is opened in the inner layer tube 1 after repeated use. If such a hole occurs, the hole cannot be easily discovered in the tube for medical instruments according to the related art since the hole cannot be visually recognized from the outside.

According to the tube for medical instruments 10 in the first embodiment, whether or not there is a hole in the inner layer tube 1 can be inspected as follows.

For example, one end of the inner layer tube 1 is blocked, and compressed air for inspection is introduced from the other end of the inner layer tube 1. If a hole is present in the inner layer tube 1, the compressed air leaking from the hole enters the interface P between the linear member 2 and the polymeric elastomer layer 4, a gap is formed between the resin coating film 29 and the polymeric elastomer layer 4. The air entering the gap presses and opens an end of the interface P at the exposed portion 2C and leaks to the outside of the tube for medical instruments 10. It is easy to enable visual recognition of the leakage of the air if the tube for medical instruments 10 is immersed in a water tank.

Therefore, in a case in which air leaks from the exposed portion 2C of the tube for medical instruments 10, it is determined that a hole is opened in the inner layer tube 1. Utilization of the tube for medical instruments 10, which has been determined to have a hole opened therein, for a medical instrument is stopped, and the tube is replaced with a non-defective product. In this manner, disadvantages in utilization of the tube for medical instruments 10 due to a defective caused by the opening of the hole are avoided.

Further, since the exposed portion 2C is formed at the outer peripheral portion of the tube for medical instruments 10 in the tube for medical instruments 10 according to the first embodiment, properties of the outer peripheral portion of the tube for medical instruments 10 can be improved in accordance with the material for the resin coating film 2B.

In a case in which a material with more satisfactory slipping properties than those of the polymeric elastomer layer 4 is used for the resin coating film 2B, for example, slippering properties at the outer peripheral portion of the tube for medical instruments 10 are improved.

In a case in which a material with higher strength than that of the polymeric elastomer layer 4 is used for the resin coating film 2B, for example, the strength at the outer peripheral portion of the tube for medical instruments 10 is improved.

According to the tube for medical instruments 10 in the first embodiment, satisfactory insertion durability is achieved even if the tube is reinforced with the metal wire 2A as described above.

### Second Embodiment

A tube for medical instruments according to a second embodiment of the present invention will be described.

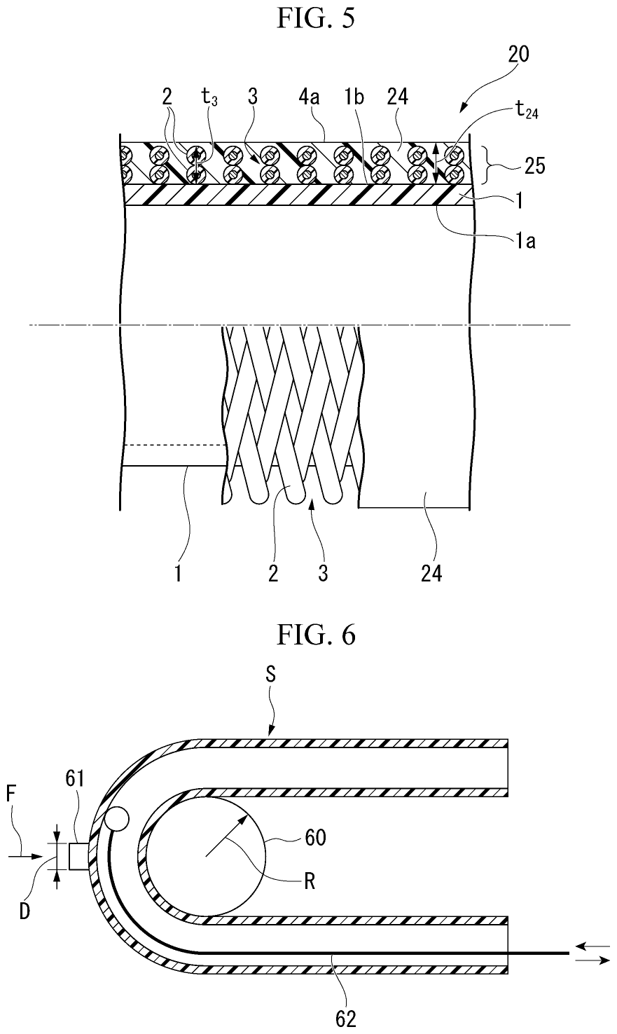

FIG. 5 is a schematic partial sectional view showing a configuration example of the tube for medical instruments according to the second embodiment of the present invention.

As shown in FIG. 5, a tube for medical instruments 20 according to the second embodiment includes an outer layer portion 25 instead of the outer layer portion 5 of the tube for medical instruments 10 according to the first embodiment.

The outer layer portion 25 includes a polymeric elastomer layer 24 instead of the polymeric elastomer layer 4 of the outer layer portion 5 according to the first embodiment.

Hereinafter, configurations, actions, and the like in the second embodiment that are different from those in the first embodiment will be mainly described.

The polymeric elastomer layer 24 is a layer obtained by changing the layer thickness of the polymeric elastomer layer 4 according to the first embodiment to such a layer thickness with which the polymeric elastomer layer 24 covers the entirety of the net-shaped body 3. Specifically, a thickness t24 of the polymeric elastomer layer 24 is thicker than the thickness t₃. Therefore, the entirety of the net-shaped body 3 according to the second embodiment is covered with the polymeric elastomer layer 24.

The tube for medical instruments 20 is manufactured in processes that are similar to those in the first embodiment other than that the application amount of the fluid body for forming the polymeric elastomer layer 24 is adjusted to the amount with which the entirety of the net-shaped body 3 is covered when the fluid body is hardened.

The tube for medical instruments 20 with the aforementioned configuration exhibits effects that are similar to those of the tube for medical instruments 10 according to the first embodiment other than that the exposed portion 2C in the first embodiment is not formed.

Therefore, according to the tube for medical instruments 20 in the second embodiment, satisfactory insertion durability is achieved even if the tube is reinforced with the metal wire 2A.

Note that although the case in which the net-shaped body 3 is configured of the linear members 2 has been described as an example in the above description of the respective embodiments, the linear members 2 may not configure the net-shaped body 3 as long as the tubes for medical instruments 10 and 20 satisfy predetermined flexibility and strength for the tube for medical instruments.

For example, the linear members 2 may be disposed by being wound in a coil shaped at the outer peripheral portion of the inner layer tube 1.

In the first embodiment, the fact that detectability of opening of a hole can be obtained since the tube for medical instruments 10 has the exposed portion 2C has been described. However, the detectability of opening of a hole is still obtained in the tube for medical instruments 20 according to the second embodiment in which the exposed portion 2C is not formed, in a case in which the polymeric elastomer layer is formed of a foaming body of sequential air bubbles.

### EXAMPLES

Next, Examples 1 to 8 of the tubes for medical instruments corresponding to the aforementioned respective embodiments will be described along with Comparative Example 1. Table 1 shows configurations of the tubes for medical instruments in the respective examples and the comparative example. However, duplicated description of conditions in the respective examples and the comparative example, such as shapes and the like, which will be described later, is omitted in Table 1. Also, reference numerals of configurations in the tubes for medical instruments corresponding to the respective embodiments are omitted in Table 1.

TABLE 1

| | | Outer layer portion | | | | | |
|---|---|---|---|---|---|---|---|
| | | Net-shaped body | | Polymeric elastomer layer | | | |
| | Inner layer | Material | Material of | | | Layer | Exposure of |
| | tube | of metal | coating | | Shore | thickness | net-shaped |
| | Material | wire | film | Material | hardness | (mm) | body |
| Example 1 | ETFE | Stainless | Polyester | Urethane-based | 65A | 0.4 | Not |
| | | steel wire | | thermoplastic | | | exposed |
| | | | | elastomer | | | |
| Example 2 | ETTE | Stainless | ETFE | Urethane-based | 65A | 0.4 | Not |
| | | steel wire | | thermoplastic | | | exposed |
| | | | | elastomer | | | |
| Example 3 | ETFE | Stainless | PTFE | Urethane-based | 65A | 0.4 | Not |
| | | steel wire | | thermoplastic | | | exposed |
| | | | | elastomer | | | |
| Example 4 | ETFE | Stainless | PFA | Urethane-based | 65A | 0.4 | Not |
| | | steel wire | | thermoplastic | | | exposed |
| | | | | elastomer | | | |
| Example 5 | PTFE | Stainless | PFA | Urethane-based | 65A | 0.4 | Not |
| | | steel wire | | thermoplastic | | | exposed |
| | | | | elastomer | | | |
| Example 6 | PFA | Stainless | PFA | Urethane-based | 65A | 0.4 | Not |
| | | steel wire | | thermoplastic | | | exposed |
| | | | | elastomer | | | |
| Example 7 | PTFE | Stainless | PFA | Fluorine rubber | 65A | 0.4 | Not |
| | | steel wire | | | | | exposed |
| Example 8 | PTFE | Stainless | PFA | Fluorine rubber | 65A | 0.25 | Exposed |
| | | steel wire | | | | | |
| Comparative | ETFE | Stainless | — | Urethane-based | 65A | 0.4 | Not |
| Example 1 | | steel wire | | thermoplastic | | | exposed |
| | | | | elastomer | | | |

### Example 1

Example 1 was an example of the tube for medical instruments 20 according to the second embodiment (see FIG. 5).

As shown in Table 1, ETFE was used as a material for the inner layer tube 1.

The inner diameter of the inner layer tube 1 in Example 1 was set to 3.2 mm, and the thickness thereof was set to 0.15 mm. The shape of the inner layer tube 1 was also applied to the inner layer tubes in Examples 2 to 8 and Comparative Example 1, which will be described later.

As shown in Table 1, the net-shaped body 3 of the outer layer portion 25 in Example 1 was configured of the linear member 2 including the metal wire 2A made of a stainless steel wire and the resin coating film 2B made of polyester resin.

The outer diameter of the metal wire 2A was set to 0.05 mm. The film thickness of the resin coating film 2B was set to 0.05 mm. The net-shaped body 3 was manufactured through twilling the linear member 2 at 30 PPI such that the number of wires thereof was 1 and the number of strokes was 16. The thickness t₃ of the net-shaped body 3 manufactured in this manner was 0.3 mm.

The outer diameter of the metal wire 2A, the film thickness of the resin coating film 2B, and the weaving conditions for the net-shaped body 3 in Example 1 were also applied to the net-shaped bodies 3 in Examples 2 to 8. The weaving conditions for the net-shaped body 3 in Example 1 were also applied to Comparative Example 1.

As shown in Table 1, a urethane-based thermoplastic elastomer as a solid body with Shore hardness of 65 A was used as a material for the polymeric elastomer layer 24 of the outer layer portion 25 in Example 1.

The layer thickness of the polymeric elastomer layer 24 was set to 0.4 mm. In this manner, the entirety of the net-shaped body 3 was covered with the polymeric elastomer layer 24.

The tube for medical instruments 20 in Example 1 as described above was manufactured by the manufacturing method described in the second embodiment.

### Examples 2 to 7

All of Examples 2 to 7 were examples of the tube for medical instruments 20 according to the second embodiment.

As shown in Table 1, the tube for medical instruments 20 in Example 2 included a configuration that was similar to that of the tube for medical instruments 20 in Example 1 other than that the material for the resin coating film 2B was changed to ETFE.

The tube for medical instruments 20 in Example 3 included a configuration that was similar to that of the tube for medical instruments 20 in Example 1 other than that PTFE was used as a material for the resin coating film 2B.

The tube for medical instruments 20 in Example 4 included a configuration that was similar to that of the tube for medical instruments 20 in Example 1 other than that PFA was used as a material for the resin coating film 2B.

The tube for medical instruments 20 in Example 5 included a configuration that was similar to that of the tube for medical instruments 20 in Example 4 than that PTFE was used as a material for the inner layer tube 1.

The tube for medical instruments 20 in Example 6 included a configuration that was similar to that of the tube for medical instruments 20 in Example 4 described above other than that PFA was used as a material for the inner layer tube 1.

The tube for medical instruments 20 in Example 7 included a configuration that was similar to that of the tube for medical instruments 20 in Example 5 other than that fluorine rubber as a solid body was used as a material for the polymeric elastomer layer 24.

### Example 8

Example 8 was an example of the tube for medical instruments 10 according to the first embodiment (see FIG. 1).

In the tube for medical instruments 10 in Example 8, the polymeric elastomer layer 4 that was made of the same material as that of the polymeric elastomer layer 24 instead of the polymeric elastomer layer 24 in Example 7 was laminated on the inner layer tube 1. The thickness of the polymeric elastomer layer 4 was 0.25 mm. Therefore, the exposed portion 2C was formed in the tube for medical instruments 10 in Example 8.

### Comparative Example 1

The tube for medical instruments in Comparative Example 1 included a configuration that was similar to that of the tube for medical instruments 20 in Example 1 other than that a stainless steel wire with an outer diameter of 0.3 mm was used instead of the linear member 2 in Example 1.

In Comparative Example 1, the linear member that formed the net-shaped body was configured of a stainless steel wire with no resin coating film.

Insertion durability, kink resistance, flexibility, and chemical resistance were evaluated using the tubes for medical instruments in Examples 1 to 8 and Comparative Example 1 as described above (hereinafter, referred to as an evaluation sample S). Further, detectability of opening of a hole was evaluated in Example 8 and Comparative Example 1.

Table 2 shows results of evaluating insertion durability, kink resistance, flexibility, and chemical resistance (evaluation results 1). Table 3 shows results of evaluating detectability of opening of a hole (evaluation results 2).

TABLE 2

| | Evaluation result 1 | | | | |
|---|---|---|---|---|---|
| | Insertion | Kink | Flexi- | Chemical | Comprehensive |
| | durability | resistance | bility | resistance | evaluation |
| Example 1 | B⁻ | B | B | B | B |
| Example 2 | B | B | B | B | B |
| Example 3 | B⁺ | B | B | B | B |
| Example 4 | B⁺ | B | B | B | B |
| Example 5 | A | B | B | B | B |
| Example 6 | A | B | B | B | B |
| Example 7 | A | B | B | A | B |
| Example 8 | A | B | B | A | B |
| Comparative | C | B | B | B | C |
| Example 1 | | | | | |

TABLE 3

| | Evaluation result 2 | | |
|---|---|---|---|
| | Sample with no | Sample with | Detectability of |
| | opening of hole | opening of hole | opening of hole |
| Example 8 | No leakage of air | Leakage of air | A |
| | | occurred | |
| Comparative | No leakage of air | No leakage of air | B |
| Example 1 | | | |

### <Evaluation Methods>

Hereinafter, the respective methods for evaluating insertion durability, kink resistance, flexibility; chemical resistance, and detectability of opening of a hole will be described.

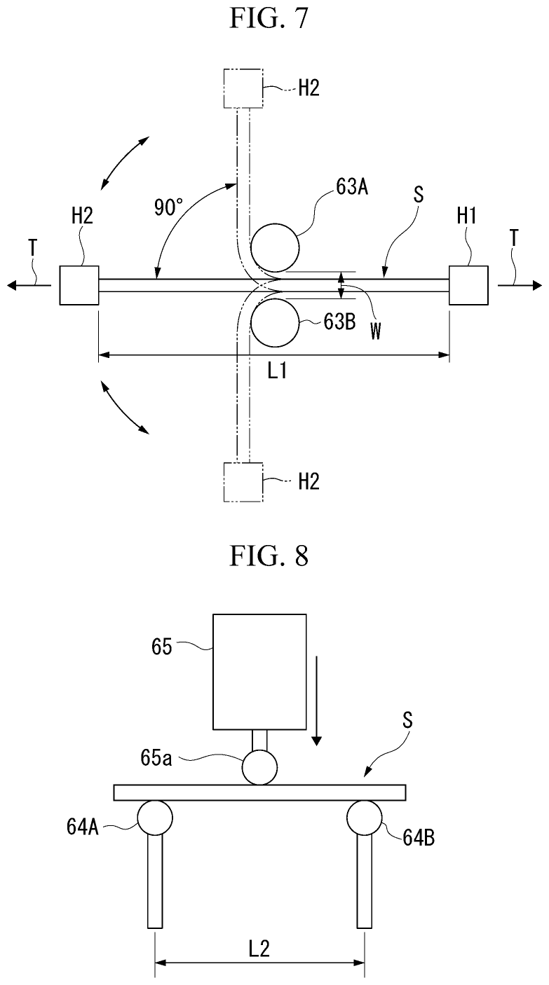

FIG. 6 is a schematic view showing a method for testing insertion durability. FIG. 7 is a schematic view showing a method for testing kink resistance. FIG. 8 is a schematic view showing a method for testing flexibility.

### <<Insertion Durability>>

In the evaluation for insertion durability, the evaluation sample S was maintained in a state in which the evaluation sample was wound and went half around a columnar winding jig 60 with a curvature radius R=9 (mm) and was bent by 180° in a plan view, as shown in FIG. 6. A circular bottom surface of a columnar pressing jig 61 with an outer diameter D=1.6 (mm) was pressed against the curved portion (a portion bent by 180° in a plan view) of the evaluation sample S with a pressing force F=2 (N). The columnar pressing jig 61 was pressed against the apex of the curved portion in the projecting shape of the evaluation sample S toward the center of the columnar winding jig 60 along a direction parallel to a linear portion of the evaluation sample S.

A biopsy forceps 62 was inserted from an end of the evaluation sample S in this state. The biopsy forceps 62 was inserted and removed such that the biopsy forceps 62 reciprocated at a hollow portion of the curved portion of the evaluation sample S at a speed of 30 mm/sec and such that a spherical portion at the tip end was brought into contact with the inner layer tube of the evaluation sample S. A biopsy forceps “FB-25K” (product name; manufactured by Olympus Corporation) was used as the biopsy forceps 62.

Whether or not a hole was opened in the inner layer tube of the evaluation sample S was inspected every 100 times on the assumption that one reciprocation of the biopsy forceps 62 was counted as one time. Whether or not a hole was opened was observed with a small-diameter endoscope inserted into the evaluation sample S. A person who evaluated recorded the number of times the biopsy forceps 62 was inserted and removed (hereinafter, referred to as the number of times before opening of a hole occurred) before opening of a hole was initially observed.

For the evaluation of insertion durability, a case in which the number of times before opening of a hole occurred exceeded 2000 was evaluated as “very good” (“A” in Table 2), a case in which the number of times before opening of a hole occurred was equal to or greater than 1600 and equal to or less than 2000 was evaluated as “rather good (“B+” in Table 2), a case in which the number of times before opening of a hole occurred was equal to or greater than 1100 and equal to or less than 1500 was evaluated as “good” (“B” in Table 2), a case in which the number of times before opening of a hole occurred was equal to or greater than 600 and equal to or less than 1000 was evaluated as “fairy good” (“B−” in Table 2), and a case in which the number of times before opening of a hole occurred was equal to or less than 500 was evaluated as “no good” (“C” in Table 2).

### <<Kink Resistance>>

For the evaluation of kink resistance, the evaluation sample S was gripped by gripping portions H1 and H2 that separate from each other by a distance L1=250 (mm) in the longitudinal direction as shown in FIG. 7. At this time, a tensile force T of 1.96 N (200 gf) was applied to the evaluation sample S between the gripping portions H1 and H2.

Further, a pair of rollers 63A and 63B with a radius of 9 mm were disposed at an interval W of 5 mm with the evaluation sample S sandwiched between the gripping portions H1 and H2 at the intermediate position thereof.

The gripping portion H1 was fixed. In other words, the gripping portion H1 was non-rotatable. The gripping portion H2 was repeatedly rotated within a range of 0°±90° when the state in which the evaluation sample S was straight was assumed to be 0° around the intermediate position. The evaluation sample S was repeatedly bent in two directions using the rollers 63A and 63B as bending support points.

A series of bending actions including rotation by +90°, returning to 0°, rotation by −90°, and returning to 0° were counted as one time, and each evaluation sample S was bent 5000 times at a speed of 29 times/minute.

After the bending of 5000 times ends, the inner diameter of the bent portion was measured with a ball gauge.

For the evaluation of kink resistance, a case in which a passing diameter of the ball gauge was equal to or greater than 3.2 was evaluated as “good” (“A” in Table 2), and a case in which the passing diameter was less than 3.2 was evaluated as “no good” (“B” in Table 2).

### <<Flexibility>>

Flexibility was evaluated on the basis of the amount of pressing force required to bend the evaluation sample S through three-point bending.

As shown in FIG. 8, two pulleys 64A and 64B with a radius of 5 mm a were disposed with an interval L2=100 (mm) at a mutually equal height in order to form support points at both ends. The evaluation sample S was placed on the pulleys 64A and 64B. A contact portion 65a of a push-pull gauge 65 was brought into contact with the evaluation sample S located at the intermediate of the pulleys 64A and 64B from the upper side. The pulleys with the radius of 5 mm were provided at the contact portion 65a. The push-pull gauge 65 was pressed into the evaluation sample S at a stroke of 40 mm toward the lower side at a speed of 20 mm/sec. At that time, a peak value of the amount of pressing force was measured by the push-pull gauge 65.

For the evaluation of flexibility, a casein which the peak value of the amount of pressing force was less than 0.8 N was evaluated as “good” (“A” in Table 2), and a case in which the peak value was equal to or greater than 0.8 N was evaluated as “no good” (“B” in Table 2).

### <<Chemical Resistance>>

For the evaluation of chemical resistance, the evaluation sample S was left in a 100% ethylene oxide gas environment at 55° C. for 60 minutes. Thereafter, the evaluation sample S was cooled to a room temperature. The amount of breaking force of the evaluation sample S was measured by pulling both ends of the evaluation sample S in the longitudinal direction with a tensile test machine at a gripping distance of 50 mm and a tensile speed of 50 mm/min.

For the evaluation of chemical resistance, a case in which the amount of breaking force was equal to or greater than 100 N was evaluated as “very good” (“A” in Table 2), a case in which the amount of breaking force was equal to or greater than 80 N and less than 100 N was evaluated as “good” (“B” in Table 2), and a case in which the amount of breaking force was less than 80 N was evaluated as “no good” (“C” in Table 2).

### <<Comprehensive Evaluation>>

For comprehensive evaluation, a case in which any of evaluations of the insertion durability, the kink resistance, the flexibility and the chemical resistance was “no good’ was evaluated as “no good” (“B” in Table 2), and the other cases were evaluated as “good” (“A” in Table 2).

### <<Detectability of Opening of Hole>>

For the evaluation of detectability of opening of a hole, “samples with no opening of a hole” manufactured in a state in which no hole was opened in the inner layer tube 1 and “samples with opening of a hole” manufactured in a state in which a hole was opened in the inner layer tube 1 in advance were created in the respective evaluation samples S in Example 8 and Comparative Example 1.

The respective evaluation samples S were immersed in an observation water tank in a state in which both ends thereof in the longitudinal direction came out to the outside of the observation water tank. In this state, and in a state in which one end of each evaluation sample S was closed, compressed air at a gauge pressure of 0.1 MPa was introduced from the other end of each evaluation sample S. A person who performed the evaluation observed whether or not air leaked depending on whether or not air bubbles occurred from the outer surface of each evaluation sample S.

For the evaluation of detectability of opening of a hole, a case in which air did not leak from the sample with no opening of a hole and air leaked from the sample with opening of a hole was evaluated as “good” (“A” in Table 3), and a case in which air did not leak from both the sample with no opening of a hole and the sample with opening of a hole was evaluated as “no good” (“B” in Table 3).

### <Evaluation Result 1>

As shown in Table 2, insertion durability of the evaluation sample S in Example 1 was evaluated as “fairly good”, insertion durability of the evaluation sample S in Example 2 was evaluated as “good’, insertion durability of the evaluation samples S in Examples 3 and 4 was evaluated as “rather good”, and insertion durability of the evaluation samples S in Examples 5 to 8 was evaluated as “very good”. The evaluation sample S in Comparative Example 1 was evaluated as “no good”.

The evaluation samples S in Examples 5 to 8 were considered to have particularly excellent insertion durability by PTFE or PFA with particularly excellent slipping properties being used as a material for the inner layer tube 1.

In Comparison of Examples 1 to 4, the evaluation samples S in Examples 3 and 4 were considered to have more excellent insertion durability than those of the evaluation samples S in Examples 1 and 2 by PTFE or PFA with particularly excellent slipping properties being used as a material for the resin coating film 2B. The insertion durability of the evaluation sample S in Example 1 was considered to be evaluated as “fairly good” by a polyester resin with poorer slipping properties than those of a fluorine resin being used as a material for the resin coating film 2B.

In regard to Comparative Example 1, it was considered that abrasion of the inner layer tube 1 was promoted since no resin coating film was formed in the linear member even if the fluorine resin was used for the inner layer tube 1. In a case in which the inner layer tube 1 was pressed with the biopsy forceps 62, for example, the linear member was not deformed, the linear member and the polymeric elastomer layer did not slip with respect to each other, and stress was thus not easily dispersed. Therefore, the abrasion of the inner layer tube 1 sandwiched between the linear member made of metal and the biopsy forceps 62 was considered to be promoted.

As shown in Table 2, both kink resistance and flexibility of the evaluation samples S in Examples 1 to 8 and Comparative Example 1 were evaluated as “good”.

Chemical resistance of the evaluation samples S in Examples 7 and 8 was evaluated as “very good, and chemical resistance of the evaluation samples S in all the examples other than Examples 7 and 8 was also evaluated as “good”.

Based on the above results, the evaluation samples S in Examples 1 to 8 were evaluated as “good”, and the evaluation sample S in Comparative Example 1 was evaluated as “no good” in the comprehensive evaluation.

### <Evaluation Result 2>

As shown in Table 3, detectability of opening of a hole of the evaluation sample S in Example 8 was evaluated as “good”, and detectability of opening of a hole of the evaluation sample S in Comparative Example 1 was evaluated as “no good”. From the evaluation results, it was recognized that satisfactory detectability of opening of a hole was achieved if a part of the net-shaped body 3 was exposed as in the evaluation sample S in Example 8. Therefore, it was recognized that the configuration according to the first embodiment was more preferred in a case in which detectability of opening of a hole was required, in particular.

Although the respective preferred embodiments of the present invention have been described above along with the respective examples, the present invention is not limited to these respective embodiments and respective examples. Addition, omission, replacement, and other changes of configurations can be made without departing from the gist of the invention.

Also, the present invention is not limited to the above description and is limited only by the scope of the claims which will be described later.

1. A tube for medical instruments comprising:
a tubular member that is made of resin;
a linear member that includes a metal wire member and a resin coating film that covers the metal wire member and that is disposed so as to surround the tubular member outside the tubular member; and
a polymeric elastomer layer that is laminated on an outer circumferential surface of the tubular member such that the polymeric elastomer layer fills in at least a part of a surface of the linear member in a circumferential direction over longitudinal direction of the linear member.

2. The tube for medical instruments according to claim 1, wherein the linear member forms a net-shaped body.

3. The tube for medical instruments according to claim 1, wherein the resin coating film is made of fluorine resin.

4. The tube for medical instruments according to claim 3, wherein the fluorine resin contains polytetrafluoroethylene.

5. The tube for medical instruments according to claim 3, wherein the fluorine resin contains tetrafluoroethylene-perfluoroalkyl vinyl ether copolymer.

6. The tube for medical instruments according to claim 1, wherein an inner circumferential surface of the tubular member is configured of fluorine resin.

7. The tube for medical instruments according to claim 1, wherein the polymeric elastomer layer contains fluorine rubber.
